(19) United States
(12) Patent Application Publication
(10) Pub. No.: US 2019/0031591 A1
(43) Pub. Date: Jan. 31, 2019

(54) METHOD FOR PREPARING MULTI-SUBSTITUTED ACRYLIC ACID COMPOUND

(71) Applicants: NATIONAL TSING HUA UNIVERSITY, Hsinchu (TW); Chang Chun Plastics Co., Ltd., Taipei (TW); Chang Chun Petrochemical Co., LTD., Taipei (TW)

(72) Inventors: Chien-Hong CHENG, HSINCHU (TW); Chen-Hsun HUNG, HSINCHU (TW); Yu-Che CHANG, HSINCHU (TW)

(21) Appl. No.: 15/805,153

(22) Filed: Nov. 7, 2017

(30) Foreign Application Priority Data
Jul. 28, 2017 (TW) 106125597

(51) Int. Cl. C07C 51/15

(52) U.S. Cl. CPC C07C 51/15

(57) ABSTRACT

A method for preparing a multi-substituted acrylic acid compound includes steps as follow. A reaction solution is provided, wherein the reaction solution includes an organometallic reagent, a nickel-containing metal catalyst, a catalyst ligand, a first solvent, and the organometallic reagent is a Grignard reagent or a Gilman reagent. An addition step is conducted, wherein an alkyne compound is mixed with the reaction solution so as to undergo an addition reaction, thus an intermediate solution is obtained. A substitution step is conducted, wherein a carbon dioxide is introduced into the intermediate solution so as to obtain the multi-substituted acrylic acid compound.

### RELATED APPLICATIONS

This application claims priority to Taiwan Application Serial Number 106125597, filed Jul. 28, 2017, which is herein incorporated by reference.

### BACKGROUND

### Technical Field

The present disclosure relates to a method for preparing a multi-substituted acrylic acid compound. More particularly, the present disclosure relates to a one-pot method for preparing the multi-substituted acrylic acid compound.

### Description of Related Art

Acrylic acid is an unsaturated carboxylic acid with a simple chemical structure and served as a common raw material used in organic synthesis method. The acrylic acid includes a double bond and a carboxyl group so that the chemical properties of the acrylic acid are quite active. It is favorable for conducting a polymerization reaction while the acrylic acids are exposed to light, heat or peroxide compounds, thus an addition reaction, an exchange reaction of functional groups, and a transesterification reaction of the acrylic acids can be conducted so as to prepare polycyclic compounds, heterocyclic compounds or acrylate compounds. In the application aspect, the acrylic acids or acrylate compounds can undergo a homopolymerization or can undergo a copolymerization with monomers such as acrylonitrile, styrene, butadiene and vinyl chloride so as to obtain polymers. The aforementioned polymers can be used as raw materials in the production of synthetic resins, synthetic fibers, superabsorbent resins, building materials and painting materials. Different preparation methods of the acrylate compounds are flourished as the growing industrial demand for the acrylic acids and derivatives thereof.

The preparation methods of the acrylic acid, such as cyanohydrin reaction method, Reppe preparation method, acrylonitrile hydrolysis method, Beta-propiolactone preparation method and propene oxidation method, have been improved for several times. Among those preparation methods, the cyanohydrin reaction preparation method is the earliest method used in the industrial production of the acrylic acids. The cyanohydrin reaction preparation method is processed by mixing a chlorohydrin and a sodium cyanide so as to obtain a cyanohydrin, and then the cyanohydrin is hydrolyzed in the presence of sulfuric acid so that the acrylic acid is obtained. Furthermore, the propene oxidation method has been improved for several times in the aspects of reaction conditions and the selection of catalysts. Because of the high yield rate of the acrylic acids, the propene oxidation method has become the major preparation method for the acrylic acids in the industrial production.

However, the aforementioned preparation methods of the acrylic acid are complicated, time-consuming and with lots of limitations in the selection of the catalysts. Furthermore, the acrylic acid contains only a carboxyl group and hydrogens which are bonded to the vinyl group, so that the application of the acrylic acid prepared by the aforementioned methods is slightly narrow. Thus, how to prepare a multi-substituted acrylic acid compound including different substituted groups and how to efficiently prepare the multi-substituted acrylic acid compound have become important goals of relevant academia and industry.

### SUMMARY

According to one aspect of the present disclosure, a method for preparing a multi-substituted acrylic acid compound includes steps as follows. A reaction solution is provided, wherein the reaction solution includes an organometallic reagent, a nickel-containing catalyst, a catalyst ligand and a first solvent, the organometallic reagent is a Grignard reagent or a Gilman reagent, the Grignard reagent is represented by Formula (ia), and the Gilman reagent is represented by Formula (ib):

R¹MgBr (ia),

R¹₂CuLi (ib),

wherein R¹ is a monovalent organic group. An addition step is conducted, wherein an alkyne compound is mixed with the reaction solution so as to undergo an addition reaction, thus an intermediate solution is obtained, and the alkyne compound is represented by Formula (ii):

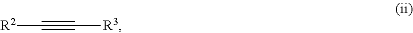

wherein R² and R³ are independently a monovalent organic group. A substitution step is conducted, wherein a carbon dioxide is introduced into the intermediate solution so as to obtain the multi-substituted acrylic acid compound, and the multi-substituted acrylic acid compound is represented by the Formula (I):

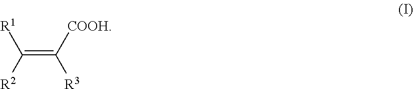

### BRIEF DESCRIPTION OF THE DRAWINGS

The present disclosure can be more fully understood by reading the following detailed description of the embodiment, with reference made to the accompanying drawing as follows:

FIG. 1 is a flow chart of the method for preparing a multi-substituted acrylic acid compound according to the present disclosure.

### DETAILED DESCRIPTION

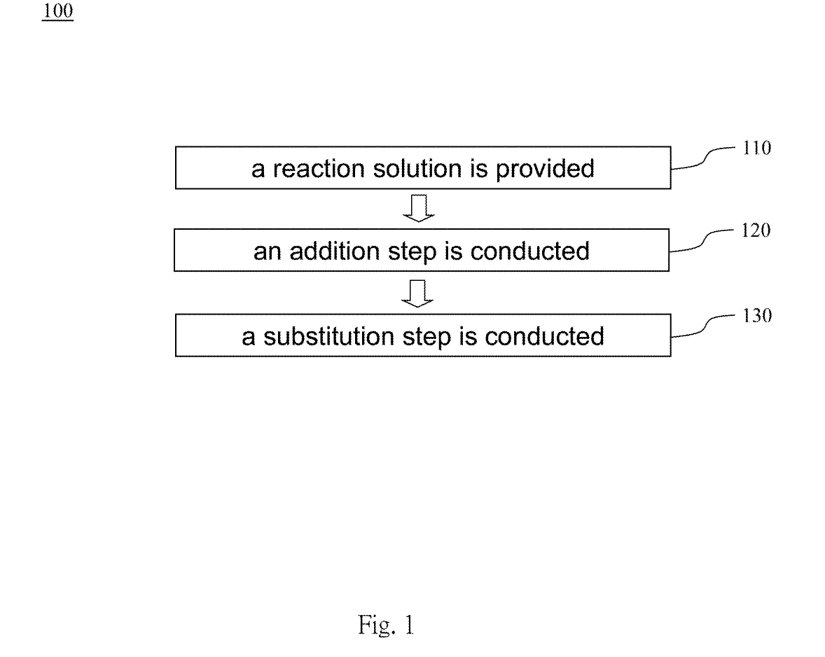

Please refer to FIG. 1, which is a flow chart of a method 100 for preparing a multi-substituted acrylic acid compound according to the present disclosure. The method 100 for preparing a multi-substituted acrylic acid compound includes Step 110, Step 120 and Step 130.

In Step 110, a reaction solution is provided, wherein the reaction solution includes an organometallic reagent, a nickel-containing catalyst, a catalyst ligand and a first solvent. The organometallic reagent is a Grignard reagent or a Gilman reagent, the Grignard reagent is represented by Formula (ia), and the Gilman reagent is represented by Formula (ib):

R¹MgBr (ia),

R¹₂CuLi (ib),

wherein R¹ is a monovalent organic group. The aforementioned nickel-containing catalyst can be a nickel halide. Furthermore, the aforementioned nickel-containing catalyst also can include a nickel halide and a first ligand, wherein the first ligand can be dimethoxyethane or bis(2-methoxyethyl) ether. Therefore, the nickel halide can be chelated by the dimethoxyethane (hereinafter, glyme) or the bis(2-methoxyethyl) ether (hereinafter, diglyme) so as to significantly increase the reaction rate of the method 100 for preparing the multi-substituted acrylic acid compound. For example, the nickel-containing catalyst can be NiBr₂.glyme, that is, the nickel-containing catalyst includes the NiBr₂ and the glyme, and the NiBr₂ is chelated by the glyme. For another example, the nickel-containing catalyst can be NiBr₂.diglyme, that is, the nickel-containing catalyst includes the NiBr₂ and the diglyme, and the NiBr₂ is chelated by the diglyme. The catalyst ligand can be a phosphine compound, wherein the phosphine compound is a derivative of a hydrogen phosphide (PH₃) that the hydrogens of the hydrogen phosphide are all substituted by monovalent organic groups, such as aliphatic groups, aryl groups or a combination thereof. More preferably, the aforementioned catalyst ligand can be triphenylphosphine (PPh₃). The catalyst ligand of the nickel-containing catalyst can enhance the catalytic activity of the nickel-containing catalyst and the reaction efficiency of the method 100 for preparing the multi-substituted acrylic acid compound. According to the organometallic reagent of the present disclosure, the R¹ of the Grignard reagent or the Gilman reagent is a monovalent organic group, such as an alkyl group or an aryl group, so that the Grignard reagent or the Gilman reagent can be served as a nucleophile. The first solvent can be an aprotic solvent, and the first solution can be but not limited to tetrahydrofuran (THF), 2-Methyltetrahydrofuran (2-MeTHF), 1,4-dioxane, 1,2-dichloroethane (DCE) and toluene (PhMe). The aforementioned aprotic solvent is a solvent which does not provide protons (H⁺) during the reaction, so that the protonation of the Grignard reagent and the Gilman reagent can be avoided, and the preparation efficiency of the multi-substituted acrylic acid compound can be enhanced.

In Step 120, an addition step is conducted, wherein an alkyne compound is mixed with the reaction solution so as to undergo an addition reaction, thus an intermediate solution is obtained, and the alkyne compound is represented by Formula (ii):

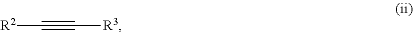

wherein R² and R³ are independently a monovalent organic group. The aforementioned addition step can be conducted at a temperature range of 50° C. to 70° C. for 30 minutes to 120 minutes. An equivalent ratio of the organometallic reagent to the alkyne compound can be 1.2:1 to 1.5:1. More preferably, the equivalent ratio of the organometallic reagent to the alkyne compound is 1.5:1. A concentration of the nickel-containing catalyst can be 5 mol % to 20 mol % based on 100 mol % of the alkyne compound. Therefore, the quantity of by-products generated during the reaction of the organometallic reagent and the alkyne compound can be reduced, so that the yield rate of the multi-substituted acrylic acid compound can be enhanced.

In Step 130, a substitution step is conducted, wherein a carbon dioxide is introduced into the intermediate solution so as to obtain the multi-substituted acrylic acid compound, and the multi-substituted acrylic acid compound is represented by the Formula (I):

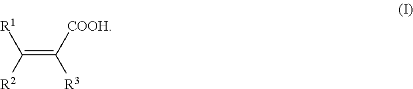

The aforementioned substitution step can be conducted at a temperature range of 15° C. to 30° C. for 20 minutes to 60 minutes.

### EXAMPLE AND COMPARATIVE EXAMPLE

### Using Grignard Reagent to Prepare Multi-Substituted Acrylic Acid Compound

The following outlines are the method for preparing the multi-substituted acrylic acid compound according to the present disclosure using a Grignard reagent.

### Example 1

a reaction solution is provided, in which the Grignard reagent is mixed with a nickel-containing catalyst, a catalyst ligand, and a first solvent so as to obtain the reaction solution. In Example 1, the Grignard reagent is CH₃MgBr, the nickel-containing catalyst is NiBr₂, the catalyst ligand is PPh₃, the first solvent is THF. An addition step is conducted, in which an alkyne compound is mixed with the reaction solution so as to undergo an addition reaction at 60° C. for 60 minutes, thus an intermediate solution is obtained, and the alkyne compound is represented by Formula (ii-1):

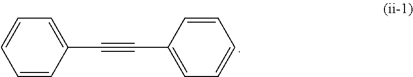 [SMILES: C(#Cc1ccccc1)c1ccccc1]

An equivalent ratio of CH₃MgBr to the alkyne compound is 1.5:1. A substitution step is conducted, in which a carbon dioxide is introduced into the intermediate solution at 25° C. for 30 minutes so as to obtain the multi-substituted acrylic acid compound of Example 1, and the multi-substituted acrylic acid compound of Example 1 is represented by the Formula (I-1):

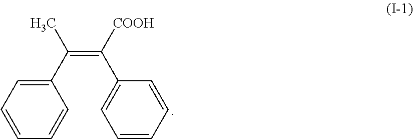 [SMILES: C/C(=C(\C(=O)O)c1ccccc1)c1ccccc1]

### Examples 2-8

the types of the nickel-containing catalyst, the concentration of the nickel-containing catalyst, the types of the catalyst ligand, and the concentration of the catalyst ligand of Example 1 can be selectively changed as shown in Table 1, and other details of Examples 2-8 are the same as that of Example 1. Moreover, the alkyne compounds and the multi-substituted acrylic acid compounds of Examples 2-8 are the same as that of Example 1, i.e., the alkyne compound is represented by Formula (ii-1) and the multi-substituted acrylic acid compounds of Examples 2-8 are represented by the Formula (I-1).

TABLE 1

| | | Con. of nickel- | | Con. of | |
|---|---|---|---|---|---|
| | Nickel- | containing | | catalyst | |
| | containing | catalyst | Catalyst | ligand | Yield rate |
| Ex. # | catalyst | (mol %) | ligand | (mol %) | (%) |
| 1 | NiBr₂ | 10 | PPh₃ | 20 | 43 |
| 2 | NiCl₂ | 10 | PPh₃ | 20 | 49 |
| 3 | NiBr₂•glyme | 10 | PPh₃ | 20 | 82 |
| 4 | NiBr₂•glyme | 5 | PPh₃ | 10 | 80 |
| 5 | NiBr₂•glyme | 5 | PPh₃ | 5 | 82 |
| 6 | NiBr₂•diglyme | 5 | PPh₃ | 10 | 76 |
| 7 | NiBr₂•diglyme | 5 | PPh₃ | 5 | 88 |
| 8 | NiBr₂•diglyme | 10 | dppe | 10 | 50 |

### Examples 9-12

the types of the catalyst ligand, the concentration of the catalyst ligand, and the types of the first solvent of Example 3 can be selectively changed as shown in Table 2, and other details of Examples 9-12 are the same as that of Example 3. Moreover, the alkyne compounds and the multi-substituted acrylic acid compounds of Examples 9-12 are also the same as that of Example 3.

TABLE 2

| | | Con. of catalyst | | Yield rate |
|---|---|---|---|---|
| Ex. # | Catalyst ligand | ligand (mol %) | First solvent | (%) |
| 9 | P(4-OMeC₆H₄)₃ | 20 | THF | 31 |
| 10 | P(4-FC₆H₄)₃ | 20 | THF | 13 |
| 11 | PPh₃ | 20 | 1,4-dioxane | 11 |
| 12 | PPh₃ | 20 | 2-MeTHF | 12 |

Please refer to Table 1, when the concentrations of the nickel-containing catalyst of Examples 1-3 are all 10 mol %, and the nickel-containing catalysts of Examples 1-3 are NiBr₂, NiCl₂ and NiBr₂.diglyme, respectively, the yield rate of Example 1 is 43%, Example 2 is 49%, and Example 3 is 82%. Therefore, it shows that the catalytic efficiency of the nickel halide can be effectively enhanced with the first ligand according to the results of Examples 1-3.

As shown in Table 1, in Examples 3-5, when the nickel-containing catalysts are NiBr₂.glyme, the catalyst ligands are PPh₃, and the concentration of PPh₃ is 20 mol % in Example 3, 10 mol % in Example 4 and 5 mol % in Example 5, the yield rates of Examples 3-5 are 82%, 80% and 82%, respectively. Therefore, it shows that the yield rate of the multi-substituted acrylic compound can be effectively enhanced with a small amount of the catalyst ligand by the results of Examples 3-5.

Furthermore, when the concentrations of the nickel-containing catalyst and the catalyst ligands are the same in Example 4 and Example 6, the first ligand of Example 4 is glyme, and the first ligand of Example 6 is diglyme, the yield rate of the multi-substituted acrylic compound of Example 4 and Example 6 are 80% and 76%, respectively. When the types of the nickel halide and the types of the catalyst ligands are the same in Example 5 and Example 7, the first ligand of Example 5 is glyme, and the first ligand of Example 7 is diglyme, the yield rates of the multi-substituted acrylic compound of Example 5 and Example 7 are 82% and 88%, respectively. Therefore, according to the results of Examples 4-7, it shows that glyme and diglyme can be served as the first ligands respectively in the present disclosure, so that the catalytic activity of the nickel halide can be significantly enhanced.

As shown in Table 1, when the nickel-containing catalyst is 10 mol % NiBr₂.diglyme, and the catalyst ligand is dppe in Example 8, the yield rate of the multi-substituted acrylic compound is 50%. When the catalyst ligands of the Example 3-7 are PPh₃, the yield rates of the multi-substituted acrylic compounds of Example 3-7 are all more than 75%. Therefore, it shows that the phosphine compound can be used as the catalyst ligand of the method for preparing a multi-substituted acrylic acid compound so as to enhance the catalytic activity of the nickel halide. More preferably, the catalytic activity of the nickel halide can be significantly enhanced when the catalyst ligand is PPh₃.

Please refer back to Table 2, when the first solvent is THF, the yield rates of the multi-substituted acrylic acid compound of Example 9 and Example 10 are 31% and 13%, respectively. Therefore, it shows that the multi-substituted acrylic acid compounds can be prepared with different phosphine compounds served as the catalyst ligand by the one-pot method for preparing the multi-substituted acrylic acid compound according to the present disclosure. Furthermore, when the first solvent of Example 11 is 1,4-dioxane and the first solvent of Example 12 is 2-MeTHF, the yield rates of the multi-substituted acrylic acid compound of Example 11 and Example 12 are 11% and 12%, respectively. It shows that the multi-substituted acrylic acid compounds can be prepared by different aprotic solvents served as the first solvent by the one-pot method for preparing the multi-substituted acrylic acid compound according to the present disclosure.

### Multi-Substituted Acrylic Acid Compound Prepared by Different Reaction Temperatures and Reaction Time

In order to estimate the effects of the temperature and the time of the addition step and the substitution step of the method for preparing a multi-substituted acrylic acid compound, Examples 13-15 and Comparative Examples 1-3 are provided. The following outlines are the method for preparing the multi-substituted acrylic acid compound according to Examples 13-15 and Comparative Examples 1-3.

### Example 13

the alkyne compound and the multi-substituted acrylic acid compound of Example 13 are the same as that of Example 1, wherein the Grignard reagent is CH₃MgBr, an equivalent ratio of CH₃MgBr to the alkyne compound is 1.5:1, the nickel-containing catalyst is the NiBr₂.diglyme, the concentration of the nickel-containing catalyst is 5 mol %, the catalyst ligand is PPh₃, the concentration of the catalyst ligand is 5 mol %, and the first solvent is DCE. Other details of the method for preparing the multi-substituted acrylic acid compound in Example 13 are the same as that of Example 1.

### Examples 14-15

the temperature and time of the addition step, and the temperature and time of the substitution step of Examples 14-15 can be selectively changed as shown in Table 3, and other details of Examples 14-15 are the same as that of Example 13.

### Comparative Examples 1-3

the temperature and time of the addition step, and the temperature and time of the substitution step of Comparative Examples 1-3 can be selectively changed as shown in Table 4, and other details of Comparative Examples 1-3 are the same as that of Example 13.

TABLE 3

| | Addition step | | Substitution step | | |
|---|---|---|---|---|---|
| | Temper- | | Temper- | | |
| | ature | Time | ature | Time | Yield rate |
| Ex. # | (° C.) | (minute) | (° C.) | (minute) | (%) |
| 13 | 60 | 60 | 15 | 30 | 73 |
| 14 | 60 | 30 | 25 | 30 | 84 |
| 15 | 60 | 120 | 25 | 30 | 88 |

TABLE 4

| | Addition step | | Substitution step | | |
|---|---|---|---|---|---|
| Compar- | Temper- | | Temper- | | |
| ative | ature | Time | ature | Time | Yield rate |
| Ex. # | (° C.) | (minute) | (° C.) | (minute) | (%) |
| 1 | Room | 60 | 25 | 30 | 10 |
| | temperature | | | | |
| 2 | 40 | 60 | 25 | 30 | 20 |
| 3 | 60 | 10 | 25 | 30 | 67 |

Please refer to Table 3, as shown in the addition step, when the temperatures of Examples 14-15 are all 60° C., the time of Example 14 is 30 minutes, and the time of Example 15 is 120 minutes, the yield rates of Example 14-15 are 84% and 88%, respectively. Therefore, it shows that the multi-substituted acrylic acid compounds can be prepared by the one-pot method according to the present disclosure with the time of the addition step in Examples 14-15.

Please refer to the Table 3 and Table 4 simultaneously, as shown in Example 15 and Comparative Example 3, when the time of the addition step is shortened, the yield rate is reduced. Furthermore, as shown in Examples 14-15 and Comparative Examples 2-3, when the time of addition step falls in the range of 30 minutes to 120 minutes, the temperature of the addition step is lowered, and the yield rate is reduced. Therefore, it shows that the multi-substituted acrylic acid compounds can be prepared by the one-pot method for preparing the multi-substituted acrylic acid compound according to the present disclosure with the time of the addition step in Examples 14-15 and Comparative Examples 2-3.

In the substitution step, as shown in Examples 13-15, when the temperatures and the times of addition step are falling in suitable ranges, the yield rate of Example 13 is 73%, the yield rate of Example 14 is 84%, and the yield rate of Example 15 is 88%. Therefore, it shows that the multi-substituted acrylic acid compounds can be prepared by the one-pot method for preparing the multi-substituted acrylic acid compound according to the present disclosure with the time of the addition step in Examples 13-15.

### Using Grignard Reagent Containing Aryl Group to Prepare Multi-Substituted Acrylic Acid Compound

In order to estimate the effects of the Grignard reagent containing aryl group in the method for preparing a multi-substituted acrylic acid compound according to the present disclosure, Examples 16-22 are provided. The following outlines are the method for preparing the multi-substituted acrylic acid compound according to the present disclosure using a Grignard reagent containing aryl group.

### Example 16

an equivalent ratio of the Grignard reagent to the alkyne compound is 1.5.1, the nickel-containing catalyst is NiBr₂.diglyme, the concentration of the nickel-containing catalyst is 5 mol %, the catalyst ligand is Tetramethylethylenediamine (TMEDA), the concentration of the catalyst ligand is 5 mol %, and the first solvent is toluene. The Grignard reagent and the multi-substituted acrylic acid compound of Example 16 are shown in Table 5, and the alkyne compound of Example 16 is represented by Formula (ii-2):

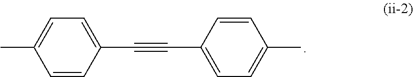

### Examples 17-22

the concentrations of the Grignard reagent, the types of the nickel-containing catalyst, the concentrations of the nickel-containing catalyst, and the types of the first solvent of Examples 17-22 can be selectively changed as shown in Table 5, and other details of Examples 17-22 are the same as that of Example 16. Moreover, the alkyne compounds of Examples 17-22 are also the same as that of Example 16, and the multi-substituted acrylic acid compounds of Examples 17-22 are shown in Table 5.

TABLE 5

| | Grignard reagent | Multi-substituted acrylic | Yield rate |
|---|---|---|---|
| Ex. # | containing aryl group | acid compound | (%) |
| 16 | 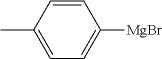 | 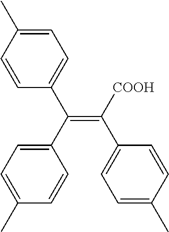 [SMILES: Cc1ccc(C(C(=O)O)=C(c2ccc(C)cc2)c2ccc(C)cc2)cc1] | 83 |
| 17 | 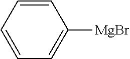 | 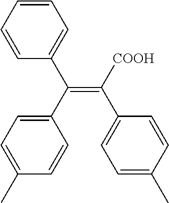 [SMILES: Cc1ccc(/C(C(=O)O)=C(/c2ccccc2)c2ccc(C)cc2)cc1] | 78 |
| 18 | 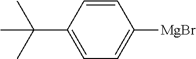 | 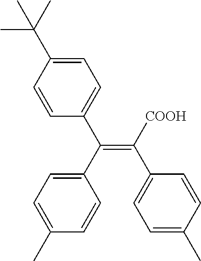 [SMILES: Cc1ccc(/C(C(=O)O)=C(\c2ccc(C)cc2)c2ccc(C(C)(C)C)cc2)cc1] | 87 |
| 19 | 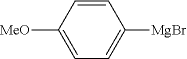 | 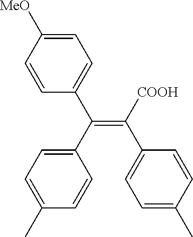 [SMILES: COc1ccc(/C(=C(\C(=O)O)c2ccc(C)cc2)c2ccc(C)cc2)cc1] | 69 |
| 20 | 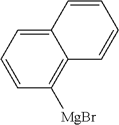 | 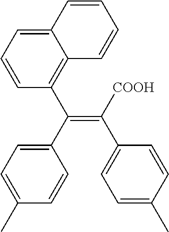 [SMILES: Cc1ccc(/C(C(=O)O)=C(\c2ccc(C)cc2)c2cccc3ccccc23)cc1] | 74 |
| 21 | 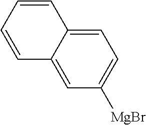 | 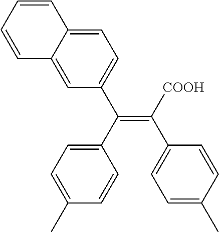 [SMILES: Cc1ccc(/C(C(=O)O)=C(\c2ccc(C)cc2)c2ccc3ccccc3c2)cc1] | 73 |
| 22 | 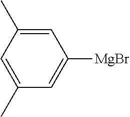 | 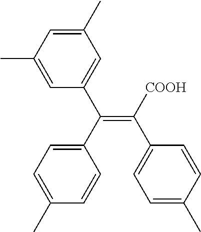 | 80 |

As shown in Table 5, the yield rate of the Example 16 is 83%, the yield rate of the Example 17 is 78%, the yield rate of the Example 18 is 87%, the yield rate of the Example 19 is 69%, the yield rate of the Example 20 is 74%, the yield rate of the Example 21 is 73%, and the yield rate of the Example 22 is 80%. Therefore, it shows that the Grignard reagents containing aryl group can catalyze the reaction for preparing the multi-substituted acrylic acid compound along with the nickel-containing catalyst and the catalyst ligand of the one-pot method for preparing the multi-substituted acrylic acid compound according to the present disclosure.

### Using Gilman Reagent to Prepare Multi-Substituted Acrylic Acid Compound

The following outlines are the method for preparing the multi-substituted acrylic acid compound according to the present disclosure using a Gilman reagent, and the method for preparing the multi-substituted acrylic acid compounds of Examples 23-24 is conducted by the steps as follow.

First, reaction solutions of Examples 23-24 are provided, in which the Grignard reagents are mixed with different types of the nickel-containing catalyst, the catalyst ligands and the first solvents so as to obtain the reaction solutions of Example 23 and Example 24, respectively. In detail, the Gilman reagent of Example 23 is (CH₃)₂CuLi, and the Gilman reagent of Example 24 is Ph₂CuLi. In Example 23, the nickel-containing catalyst is NiBr₂.diglyme, the concentration of the nickel-containing catalyst is 5 mol %, the catalyst ligand is PPh₃, the concentration of the catalyst ligand is 5 mol %, and the first solvent is THF. The nickel-containing catalyst, the concentration of the nickel-containing catalyst, the catalyst ligand, the concentration of the catalyst ligand and the first solvent of Example 24 are the same as that of Example 23.

Second, an addition step is conducted, in which an alkyne compound is mixed with the reaction solution so as to undergo an addition reaction at 60° C. for 60 minutes, and an equivalent ratio of the Gilman reagent to the alkyne compound is 1.5:1, thus an intermediate solution is obtained. The alkyne compounds of Examples 23-24 are same as that of Example 16, which is represented by Formula (ii-2):

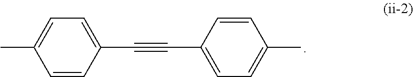

Latest, a substitution step is conducted, in which a carbon dioxide is introduced into the intermediate solution at 25° C. for 30 minutes so as to obtain the multi-substituted acrylic acid compounds of Examples 23-24, respectively. In detail, the multi-substituted acrylic acid compound of Example 23 is represented by the Formula (1-2), and the multi-substituted acrylic acid compound of Example 24 is represented by the Formula (1-3):

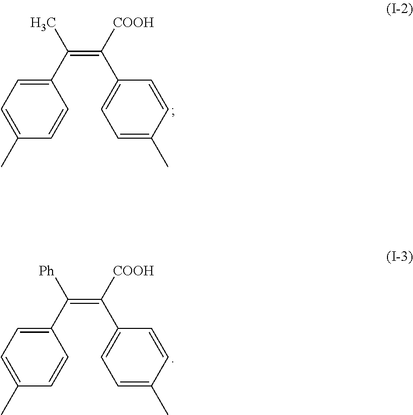 [SMILES: C/C(=C(\C(=O)O)c1ccc(C)cc1)c1ccc(C)cc1 ; Cc1ccc(/C(C(=O)O)=C(/c2ccccc2)c2ccc(C)cc2)cc1]

As the results, the yield rate of the multi-substituted acrylic acid compound of Example 23 is 15%, and the yield rate of the multi-substituted acrylic acid compound of Example 24 is 42%. Therefore, it shows that the multi-substituted acrylic acid compounds can be prepared by using the Gilman reagent in the one-pot method for preparing the multi-substituted acrylic acid according to the present disclosure.

### Stereochemical Structure of Multi-Substituted Acrylic Acid Compound

Because of the electronic effect and the ortho-directing effect generated during the reaction, the stereochemical structure of the multi-substituted acrylic acid compounds prepared by the method according to the present disclosure is usually represented by the aforementioned Formula (I). The electronic effect is using the difference degrees between the electron donating force and the electron withdrawing force of the functional group to change the charge distribution of the alkyne compound, so that a selective difference of the reaction is obtained. The ortho-directing effect is using the intramolecular force between the ortho-functional group in a benzene compound and the nickel-containing catalyst to obtain a selective difference force of the asymmetric alkyne compounds during the reaction. According to the previous literature and the results of the identification of the products, the reaction type of the method for preparing a multi-substituted acrylic acid compound according to the present disclosure is cis-addition reaction, that is, the substituted groups R² and R³ of the alkyne compound will be represented in a cis-configuration in the multi-substituted acrylic acid compound. The reaction properties of the multi-substituted acrylic acid compound with cis-configuration are more active than the multi-substituted acrylic acid compound with trans-configuration, so that the multi-substituted acrylic acid compound with cis-configuration is favorable for processing the organic polymerization.

In order to illustrate stereochemical structures of the multi-substituted acrylic acid compounds prepared by the method according to the present disclosure, Examples 25-33 are provided. In Examples 25-33, the types of the organometallic reagent, the concentrations of the organometallic reagent, the types of the nickel-containing catalyst, the concentrations of the nickel-containing catalyst, the types of the catalyst ligand, and the concentrations of the catalyst ligand are the same as that of Example 7. The alkyne compounds and the multi-substituted acrylic acid compounds of Examples 25-33 are shown in Table 6. Moreover, the alkyne compounds used in Examples 25-30 are symmetric alkyne compounds, in which the substituted group R² and R³ of the alkyne compound are the same, and the alkyne compounds used in Examples 31-33 are asymmetric alkyne compounds, in which the substituted group R² and R³ of the alkyne compound are different to each other.

TABLE 6

| | | Multi-substituted | |
|---|---|---|---|
| | | acrylic acid compound | Percentage |
| Ex. # | Alkyne compound | with cis-configuration | (%) |
| 25 | 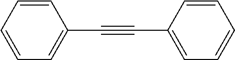 [SMILES: C(#Cc1ccccc1)c1ccccc1] | 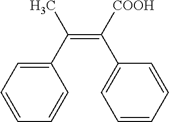 [SMILES: C/C(=C(\C(=O)O)c1ccccc1)c1ccccc1] | 81 |
| 26 | 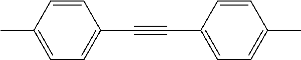 | 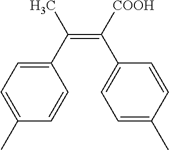 [SMILES: C/C(=C(\C(=O)O)c1ccc(C)cc1)c1ccc(C)cc1] | 83 |
| 27 | 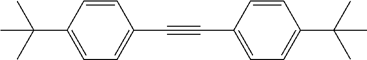 [SMILES: CC(C)(C)c1ccc(C#Cc2ccc(C(C)(C)C)cc2)cc1] | 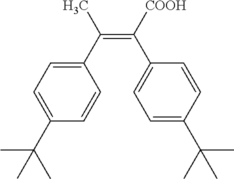 [SMILES: C/C(=C(\C(=O)O)c1ccc(C(C)(C)C)cc1)c1ccc(C(C)(C)C)cc1] | 70 |
| 28 | 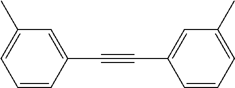 [SMILES: Cc1cccc(C#Cc2cccc(C)c2)c1] | 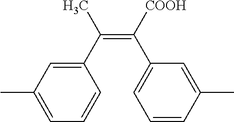 | 69 |
| 29 | 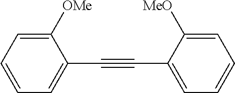 [SMILES: COc1ccccc1C#Cc1ccccc1OC] | 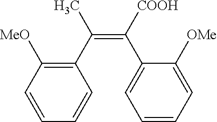 [SMILES: COc1ccccc1/C(C)=C(/C(=O)O)c1ccccc1OC] | 83 |
| 30 | 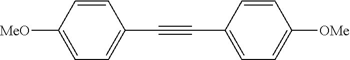 [SMILES: COc1ccc(C#Cc2ccc(OC)cc2)cc1] | 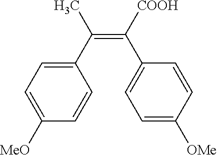 [SMILES: COc1ccc(/C(C)=C(/C(=O)O)c2ccc(OC)cc2)cc1] | 93 |
| 31 | 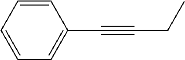 | 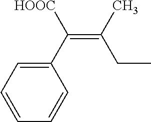 | 69 |
| 32 | 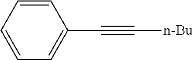 | 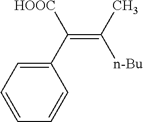 | 76 |
| 33 | 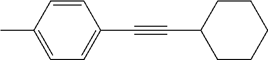 | 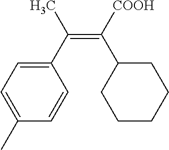 [SMILES: C/C(=C(\C(=O)O)C1CCCCC1)c1ccc(C)cc1] | 63 |

As shown in Table 6, no matter the alkyne compound is a symmetric alkyne compound or an asymmetric alkyne compound, the yield rates of the multi-substituted acrylic acid compound with cis-configuration of Examples 25-33 are all higher than the yield rates of the multi-substituted acrylic acid compounds with trans-configuration. Therefore, it shows that the multi-substituted acrylic acid compound with cis-configuration can be prepared by the one-pot method for preparing the multi-substituted acrylic acid compound according to the present disclosure.

### Using Different Equivalent Ratios of Organometallic Reagent to Prepare Multi-Substituted Acrylic Acid Compound

In order to estimate the yield rates of the multi-substituted acrylic acid compound prepared with different equivalent ratios of the organometallic reagent of the method according to the present disclosure, Example 34 is provided. In Example 34, the type of the organometallic reagent, the type of the nickel-containing catalyst, the concentration of the nickel-containing catalyst, the type of the catalyst ligand, and the concentration of the catalyst ligand are the same as that of Example 19. In detail, the organometallic reagent of Example 34 is CH₃MgBr, and an equivalent ratio of the organometallic reagent to the alkyne compound of Example 34 is 1.2:1. The alkyne compound and the multi-substituted acrylic acid compound of Example 34 is shown in Table 7.

TABLE 7

| | | Equivalent | Multi- | |
|---|---|---|---|---|
| | | ratio of the | substituted | Yield |
| Ex. | Alkyne | organometallic | acrylic acid | rate |
| # | compound | reagent | compound | (%) |
| 34 | 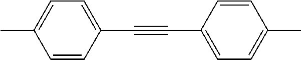 | 1.2 | 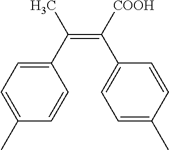 [SMILES: C/C(=C(\C(=O)O)c1ccc(C)cc1)c1ccc(C)cc1] | 85 |

As shown in Table 7, the yield rate of the multi-substituted acrylic acid compound of Example 34 is 85%. Therefore, it shows that the multi-substituted acrylic acid compound also can be prepared by the one-pot method for preparing the multi-substituted acrylic acid compound according to the present disclosure with different equivalent ratios of the organometallic reagent to the alkyne compound.

According to the aforementioned Examples, the present disclosure has the advantages described bellowing:

First, by using proper nickel-containing catalyst, catalyst ligand and the first solvent, the multi-substituted acrylic acid compound can be prepared by the one-pot method for preparing a multi-substituted acrylic acid compound including a continuous two-stage reaction. Therefore, the preparation time and the production costs can be significantly reduced.

Second, the functional groups of the multi-substituted acrylic compounds can be adjusted by the method for preparing a multi-substituted acrylic acid compound according to the present disclosure, so that the multi-substituted acrylic compounds with different functions are obtained. Therefore, the aforementioned multi-substituted acrylic compounds can be further applied to different uses, and the application of the multi-substituted acrylic compound can be expanded.

Third, the multi-substituted acrylic acid compound with cis-configuration can be prepared by the method for preparing a multi-substituted acrylic acid compound according to the present disclosure, and it is favorable for processing the organic polymerization.

Fourth, because the carbon dioxide is one of the reactants of the method for preparing a multi-substituted acrylic acid compound, the method according to the present disclosure can be further applied to fixed the carbon dioxide so as to reduce the concentration of the carbon dioxide in the atmosphere and further facilitate the development of the green industry.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure according to the present disclosure without departing from the scope or spirit of the disclosure. In view of the foregoing, it is intended that the present disclosure cover modifications and variations of this disclosure provided they fall within the scope of the following claims.

What is claimed is:

1. A method for preparing a multi-substituted acrylic acid compound, comprising:
providing a reaction solution, wherein the reaction solution comprises an organometallic reagent, a nickel-containing catalyst, a catalyst ligand and a first solvent, the organometallic reagent is a Grignard reagent or a Gilman reagent, the Grignard reagent is represented by Formula (ia), and the Gilman reagent is represented by Formula (ib):
R¹MgBr (ia),
R¹₂CuLi (ib),
wherein R¹ is a monovalent organic group;
conducting an addition step, wherein an alkyne compound is mixed with the reaction solution so as to undergo an addition reaction, thus an intermediate solution is obtained, and the alkyne compound is represented by Formula (ii): 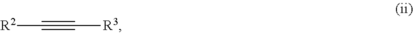
wherein R² and R³ are independently a monovalent organic group; and
conducting a substitution step, wherein a carbon dioxide is introduced into the intermediate solution so as to obtain the multi-substituted acrylic acid compound, and the multi-substituted acrylic acid compound is represented by the Formula (I):

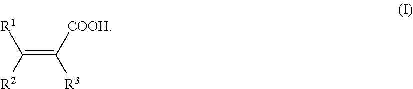

2. The method for preparing the multi-substituted acrylic add compound of claim 1, wherein the nickel-containing catalyst is a nickel halide.

3. The method for preparing the multi-substituted acrylic acid compound of claim 1, wherein the nickel-containing catalyst comprises a nickel halide and a first ligand.

4. The method for preparing the multi-substituted acrylic acid compound of claim 3, wherein the first ligand is dimethoxyethane or bis(2-methoxyethyl) ether.

5. The method for preparing the multi-substituted acrylic acid compound of claim 1, wherein a concentration of the nickel-containing catalyst is 5 mol % to 20 mol % based on 100 mol % of the alkyne compound.

6. The method for preparing the multi-substituted acrylic acid compound of claim 1, wherein the catalyst ligand is a phosphine compound.

7. The method for preparing the multi-substituted acrylic acid compound of claim 6, wherein the catalyst ligand is triphenylphosphine.

8. The method for preparing the multi-substituted acrylic acid compound of claim 1, wherein the first solvent is an aprotic solvent.

9. The method for preparing the multi-substituted acrylic add compound of claim 8, wherein the first solvent is tetrahydrofuran.

10. The method for preparing the multi-substituted acrylic acid compound of claim 1, wherein the addition step is conducted at a temperature range of 50° C. to 70° C. for 30 minutes to 120 minutes.

11. The method for preparing the multi-substituted acrylic acid compound of claim 1, wherein the substitution step is conducted at a temperature range of 15° C. to 30° C. for 20 minutes to 60 minutes.

12. The method for preparing the multi-substituted acrylic acid compound of claim 1, wherein an equivalent ratio of the organometallic reagent to the alkyne compound is 1.2:1 to 1.5:1.

13. The method for preparing the multi-substituted acrylic acid compound of claim 1, wherein an equivalent ratio of the organometallic reagent to the alkyne compound is 1.5:1.
